(19) United States
(12) Patent Application Publication
(10) Pub. No.: US 2005/0013886 A1
(43) Pub. Date: Jan. 20, 2005

(54) Cigarette substitute and aid to quit tobacco use

(76) Inventors: Marc Charles Dawe, Tonopah, NV (US); Charles Bertolino, Tonopah, NV (US)

Correspondence Address: KENNETH D' ALLESANDRO;SIERRA PATENT GROUP, LTD., PO BOX 6149, STATELINE, NV, 89449, US

(21) Appl. No.: 10/393,785

(22) Filed: Jul. 18, 2003

(51) Int. Cl. A61K 35/78

(52) U.S. Cl. 424/769

(57) ABSTRACT

The disclosed device is directed towards a device for the cessation of tobacco use comprising a body having a core and a surface disposed on the core. The body is substantially cylindrical having a diameter of about five sixteenths of an inch and a length of about three and one-quarter inches. A composition of nicotine is disposed in the body.

### BACKGROUND

The present disclosure relates to the field of tobacco addiction aids. In particular, the present disclosure relates to a device and method for the cessation of tobacco use. The chemical and mechanical habits of smoking tobacco have long been documented as continuing a harmful and addictive practice. Part of the addiction to tobacco includes both the chemical addiction and repetitive physical habit addiction. Prior art tobacco use cessation aids have provided relief from the chemical addictions. Although the prior art provides the user relief for the nicotine cravings, the prior art overlooks the repetitive physical addiction. The prior art fails to address the physical manipulation of the cigarette as it is smoked, in trying to aid the smoker to give up the habit. The physical habit addiction can include other tobacco uses such as, chew, dip snuff, etc.

### SUMMARY

The disclosed device is directed towards a device for the cessation of tobacco use comprising a body having a core and a surface disposed on the core. The body is substantially cylindrical having a diameter of about five sixteenths of an inch and a length of about three and one-quarter inches. A composition of nicotine is disposed in the body.

The disclosed device is directed towards a device for the cessation of tobacco use comprising a container having a base coupled to a side wall coupled to a removable top. The base, side wall and removable top define an interior and an exterior. At least one cigarette substitute chewing stick is removably disposed in the interior.

The cigarette substitute can be made of birch wood, containing the equivalent nicotine content of one cigarette per stick, half the nicotine content of one cigarette per stick, or a nicotine free stick. This nicotine would not be inhaled, it would be absorbed orally, in the same manner it is absorbed when chewing tobacco, as the user chews and orally manipulates the stick, much as one may chew on a toothpick.

The actual physical mechanical motions required in using this device, along with the slower nicotine absorption rate, will give a smoker trying to give up his habit the extra edge he needs to quit. As he becomes accustom to eliminating only the smoking part of his habit, the slower consumption rate of the nicotine will help him to go from the full nicotine content product, to the half nicotine content product, to the nicotine free product, when the manipulation of the product alone is enough to keep from lighting up again.

### BRIEF DESCRIPTION OF THE DRAWINGS

FIG. 1 is a side and end view of an exemplary cigarette substitute chewing stick.

FIG. 2 is a front view of an exemplary pack of cigarette substitute chewing sticks.

### DETAILED DESCRIPTION

Cigarette substitute chewing sticks can be made of birch wood and have the same dimensions as a “king size” cigarette, three and one quarter inches long by five sixteenths inches diameter. Twenty sticks would come in a package, mirroring the packaging of cigarettes. In a preferred embodiment, three different potencies of nicotine composition can be available; about one milligram of nicotine per stick, about one half milligram of nicotine per stick, or no nicotine. This product can also be available with different flavor enhancement compositions, such as mint, wintergreen or cherry.

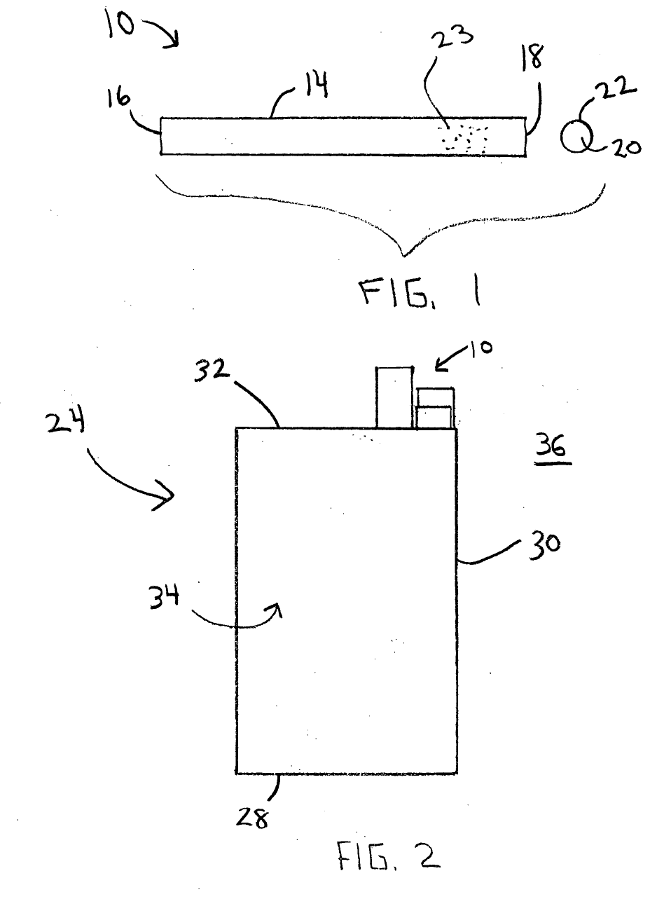

Referring to FIG. 1, a side view and an end view of an exemplary cigarette substitute chewing stick is illustrated. The cigarette substitute chewing stick or simply chewing stick 10 includes a body 14 having a first end 16 and a second end 18 opposite the first end 16. The body 14 includes a core 20 disposed in an exterior surface 22. In a preferred exemplary embodiment, the body 14 is substantially cylindrical similar in shape to a cigarette. The dimensions of the body 14 from the first end 16 to the second end 18 can be three and one quarter inches long and the diameter of the core 20 and exterior surface 22 can be five sixteenths of an inch. The dimensions of the body 14 can be equal to the dimensions of cigarettes of varying sizes in another exemplary embodiment. Nicotine 23 is inserted in the body 14, in a preferred embodiment, as a liquid that dries and remains stable until becoming soluble through saliva.

Referring to FIG. 2, a front view of an exemplary pack of cigarette substitute chewing sticks 10 is illustrated. The pack or package 24 of cigarette substitute chewing sticks includes a container 26 having a base 28 coupled to a side wall 30 and coupled to a top 32. The base 28, side wall 30 and top 32 define an interior 34 and an exterior 36. FIG. 2 illustrates three cigarette substitute chewing sticks 10 partially extending through the top 32 and partially disposed in the interior 34 and exterior 36. The pack 24 can contain a predetermined quantity of cigarette substitute chewing sticks 10. In an exemplary embodiment, twenty cigarette substitute chewing sticks 10 can be contained in the interior 34 of the pack 24. The pack 24 can resemble the tactile sensation of an ordinary pack of cigarettes (not shown).

In use the cigarette substitute chewing stick 10 and pack 24 can be similar to an ordinary cigarette use. The pack 24 size and tactile sense can resemble the pack of cigarettes. The user can open the pack 24 and dispense a single cigarette substitute chewing stick 10 in a similar manner to an ordinary cigarette. The pack 24 can be tapped and manipulated resembling the manner of use for an ordinary cigarette pack. The cigarette substitute chewing stick 10 can be placed in the mouth and held in the fingers to replicate the use of an ordinary cigarette. The cigarette substitute chewing stick 10 is not to be ignited or combusted in any manner. Thus, the use of the cigarette substitute chewing stick 10 eliminates the need for an ignition source, such as matches or lighter. The nicotine 23 is made soluble by the users saliva and released into the mouth of the user. The user can manipulate the cigarette substitute chewing stick 10 as well as chew or simply hold the cigarette substitute chewing stick 10 in the mouth, mimicking the use of an ordinary cigarette.

While embodiments and applications of this invention have been shown and described, it would be apparent to those skilled in the art that many more modifications than mentioned above are possible without departing from the inventive concepts herein. The invention, therefore, is not to be restricted except in the spirit of the appended claims.

1. A device for the cessation of tobacco use comprising:
a body having a core and a surface disposed on said core, said body being substantially cylindrical having a diameter of about five sixteenths of an inch and a length of about three and one-quarter inches; and
a composition of nicotine disposed in said body.

2. The device of claim 1 wherein said body comprises birch wood.

3. The device of claim 1 wherein said nicotine composition is soluble in saliva.

4. The device of claim 1 wherein said nicotine composition comprises an equivalent quantity of nicotine in one cigarette.

5. The device of claim 1 wherein said nicotine composition comprises at least one of about one milligram of nicotine and about one half milligram of nicotine.

6. The device of claim 1 further comprising:
a flavor enhancement composition disposed in said body.

7. The device of claim 6 wherein said body includes said enhancement composition in the absence of said nicotine composition.

8. The device of claim 6 wherein said flavor enhancement composition is selected from the group consisting of mint, wintergreen and cherry.

9. A device for the cessation of tobacco use comprising:
a container having a base coupled to a side wall coupled to a removable top, said base, side wall and removable top defining an interior and an exterior; and
at least one cigarette substitute chewing stick removably disposed in said interior.

10. The device of claim 9 wherein said container is configured to resemble the tactile features of an ordinary pack of cigarettes.

11. The device of claim 9 wherein said container is configured to contain twenty said cigarette substitute chewing sticks.

12. The device of claim 9 wherein said container is configured having dimensions of an ordinary pack of cigarettes.

13. The device of claim 9 wherein said container is configured to contain at least one of said cigarette substitute chewing stick having about one milligram of nicotine, said cigarette substitute chewing stick having about one half milligram of nicotine, and said cigarette substitute chewing stick having no nicotine.

14. The device of claim 9 wherein said container is configured to contain at least one of said cigarette substitute chewing stick having a flavor enhancement composition of one of mint, wintergreen and cherry.

15. A method of using a device for the cessation of tobacco use comprising:
handling a pack of cigarette substitute chewing sticks, said pack including a container having a base coupled to a side wall coupled to a removable top, said base, side wall and removable top defining an interior and an exterior, at least one cigarette substitute chewing stick removably disposed in said interior;
removing said at least one cigarette substitute chewing stick;
manipulating said at least one cigarette substitute chewing stick;
inserting said at least one cigarette substitute chewing stick into a mouth; and
extracting a portion of a nicotine composition disposed in said at least one cigarette substitute chewing stick.

16. The method of claim 15 further comprising:
extracting a portion of a flavor enhancement composition disposed in said at least one cigarette substitute chewing stick.

17. The method of claim 16, wherein said flavor enhancement composition disposed in said at least one cigarette substitute chewing stick is at least one of mint, wintergreen and cherry.

18. The method of claim 17 wherein said nicotine composition disposed in said at least one cigarette substitute chewing stick is variable for each of said at least one cigarette substitute chewing stick.

19. The method of claim 17 wherein said nicotine composition disposed in said at least one cigarette substitute chewing stick is one of about one milligram, about one half milligram and zero milligrams.
